(19) United States
(12) Patent Application Publication
(10) Pub. No.: US 2004/0028721 A1
(43) Pub. Date: Feb. 12, 2004

(54) Film for active ingredients dermal and transdermal administration

(76) Inventors: Paolo Colombo, Parma (IT); Pier Luigi Catellani, Reggio Emilio (IT); Cristina Padula, Trivigno (IT); Patrizia Santi, Salsomggiore (IT); Gaia Colombo, Parma (IT)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/399,367

(22) PCT Filed: Oct. 11, 2001
(86) PCT No.: PCT/EP01/11768

(30) Foreign Application Priority Data
Oct. 13, 2000 (IT) MI2000A002216

(51) Int. Cl. A61K 9/70

(52) U.S. Cl. 424/443

(57) ABSTRACT

Single-layer film for active ingredients dermal and transdermal administration containing at least an active ingredient, a film-forming agent, and a hydrophilic adhesive polymer.

### FIELD OF THE INVENTION

The present invention refers to a single-layer film for active ingredients dermal and transdermal administration and to a method for the preparation of same.

### STATE OF THE ART

In the last few years, active ingredients dermal and transdermal administration has been given a substantial boost thanks to the development of new arrangements—in particular dermal and transdermal sticking plasters—for active ingredients release to the skin.

Said sticking plasters usually consist of several layers of various materials, superimposed in the following sequence:

1. the backing layer, which is an essential element of sticking plasters found in commerce. It acts as a plaster “skeleton” and provides the suitable consistency for plaster handling and positioning on the skin. It is a transparent or opaque thin plastic film, usually occlusive to favour epidermis hydration. It must exhibit particularly good qualities of flexibility and resistance;

2. the drug depot, which is solid or semisolid or liquid and contains the active ingredient in the dispersed or dissolved state;

3. the membrane for active ingredient controlled release: once the sticking plaster has been applied, the membrane interposes between the drug depot and the skin and serves to control the active ingredient release rate;

4. the adhesive layer, which facilitates the plaster adhesion to the skin. It must secure the plaster contact with the skin surface and, at the same time, be permeable by the drug.

In addition to the aforementioned functional layers, the sticking plaster includes a protective layer consisting of a plastic sheet, coated with silicone polymers or fluoropolymers, which provide anti-adherent properties. Said layer protects the active ingredient and prevents unwanted adhesion during plaster handling and storage. The protective layer is removed immediately prior to the plaster use and, therefore, has no therapeutic function.

The sticking plasters found in commerce consist of all, or some, layers listed above. By way of example, the so-called “reservoir plasters” consist of all said layers, whereas other plasters, such as those referred to as “matrix”, include all elements excepting the membrane. The simplest sticking plasters marketed today are the so-called “drug in adhesive” ones, which consist of the backing and a drug/adhesive mixture exerting the double function of drug depot and adhesive layer. In “drug in adhesive” plasters, the drug is directly dispersed in the adhesive. Therefore, to our knowledge, in addition to the protective sheet, at least two layers are to be coupled in a dermal or transdermal sticking plaster.

Once applied, all sticking plasters exhibit a multi-layered structure in which the lower layer acts as an adhesive and the upper one as a support.

Dermal and transdermal sticking plasters are generally manufactured by lamination, whereby the single layers that already possess the required properties are superimposed one on top of the other. This method is rather complex and expensive as it requires preformed materials and elaborate procedures for layers coupling, and involves considerable material losses.

Furthermore, since the adhesives commonly used in plasters manufacture consist of water-insoluble polymers, the process must be carried out in the presence of organic solvents, e.g. ethyl acetate or toluene, which pose considerable safety problems.

Two-layered sticking plasters, i.e. the “drug in adhesive” ones, are prepared by simpler procedures, which envisage the spreading of the adhesive solution or viscous suspension on the preformed backing, followed by drying. However, also these sticking plasters suffer from the inconveniences caused by the presence of organic-based adhesives.

As may be inferred from the above description, the technology for the manufacture of transdermal sticking plasters brings about considerable disadvantages, especially due to the manufacture complexity and to the use of organic solvents. Therefore, the need for an arrangement for active ingredients dermal and transdermal administration, manufactured by simple and little expensive procedures, which, furthermore, do not require organic solvents, is acknowledged.

### SUMMARY

The Applicant has surprisingly found a new arrangement, in the form of a thin film, for active ingredients dermal and transdermal administration. The three elements that constitute the traditional sticking plaster, i.e. backing, drug depot, and adhesive layer, become indistinguishable and form a single element self-supporting. Said arrangement can be prepared by simple and little expensive procedures, which may use water-soluble polymers. Furthermore, being permeable by water, it may be easily tolerated and used for iontophoretic applications.

### DESCRIPTION OF THE FIGURES

FIG. 1 is a bottom view (a) and a side view (b) of one of the possible embodiments of the film of the invention (I), supported by an antistick protective layer (II).

FIG. 2 schematically illustrates the procedure for applying the film of the present invention to moistened skin.

FIG. 3 shows the cumulative average amount of lidocaine (μg) per mg of stratum corneum after application of the single-layer film to moistened skin (I), of a commercial lidocaine formulation (II), of the single-layer film to non-moistened skin (III) or of the single-layer film to moistened skin with iontophoretic application (IV).

FIG. 4 shows the average lidocaine distribution in the stratum corneum vs. the distance from the dermal surface: adhesive tapes 1-5, 6-10 and 11-15 include stratum corneum fragments localised at a gradually increasing distance from the surface.

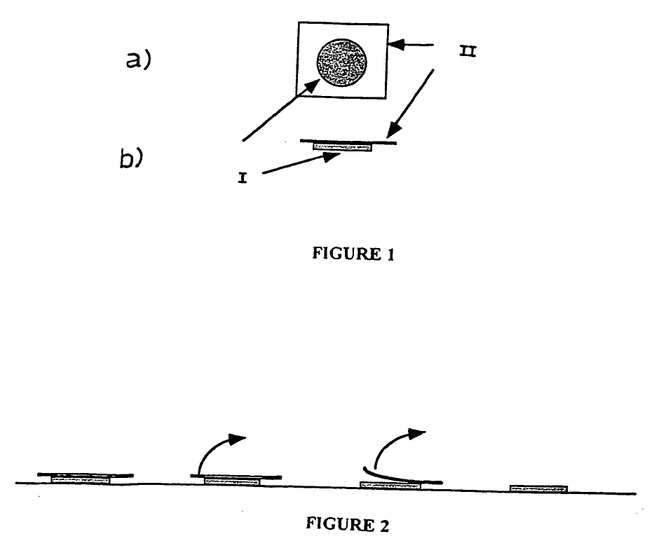

### DETAILED DESCRIPTION OF THE INVENTION

It is an object of the present invention to provide a single-layer film for active ingredients dermal and transdermal administration, comprising at least an active ingredient, a film-forming agent and a hydrophilic adhesive polymer.

The active ingredient may be in the dissolved or dispersed state.

The film of the present invention is useful for dermal or transdermal administration of any hydrophilic or lipophilic substance exerting a pharmacological or cosmetic action. Substances particularly suitable for administration through the film of the present invention are drugs for dermatologic use, e.g. topical anaesthetics, antimycotic drugs, antiinflammatory agents, cortisone-based drugs, antiviral agents, antineoplasia drugs, antihistamine drugs, antipsoriasis agents and antibiotics; drugs that may be administered by the transdermal way, e.g. nitroglycerin, sex hormones and nicotine; active ingredients for cosmetic use, e.g. keratolytics, keratoplastics, agents for the treatment of seborrhea, acne and depigmentation, disinfectants, and sebonormalisers.

The film-forming agent is preferably selected from the group consisting of ethylcellulose, acrylic and methacrylic polymers in an aqueous dispersion, and polyvinyl alcohol. According to the present invention by “acrylic and methacrylic polymers” is meant neutral acrylic and methacrylic polymers, i.e. acrylic and methacrylic polymers not having cationic or anionic charge, such as neutral copolymer based on ethyl acrylate and methyl methacrylate.

Preferably, the film-forming agent is polyvinyl alcohol having a molecular weight of 500 to 100,000 Da, especially of 49,000 to 72,000 Da. Said polyvinyl alcohol has a hydrolysis degree ranging preferably from 80% to 99%, especially from 85 to 89%.

Preferably, the hydrophilic adhesive polymer is selected from the group consisting of polyvinylpyrrolidone, tragacanth, gum arabic, karaya. xanthan gum, guar gum, acrylic and methacrylic adhesives, carrageenan and rosin. Particularly preferred are polyaminomethacrylates, preferably Eudragit E100, and tragacanth. Water solutions of Eudragit E100, mixed with lauric acid, adipic acid and glycerin are available under the trademark Plastoid E 35 L, M and H from Röhm GmbH, Darmstadt, Germany.

In the film of the invention, particularly preferred is the combination of polyvinyl alcohol having a molecular weight of 500 to 100,000. Da, especially of 49,000 to 72,000 Da, as film-forming agent, with a polyaminomethacrylate, preferably Eudragit E100, or tragacanth, as a hydrophilic adhesive polymer. Preferably, said polyvinyl alcohol has a hydrolysis degree ranging from 80 to 99%, especially from 85 to 89%.

The single-layer film of the invention optionally comprises absorption promoters and/or humectants and/or plasticisers, e.g. glycerin, ethyl alcohol, propylene glycol, polyethylene glycol having a molecular weight ranging from 400 to 6,000, sorbitol, phospholipids, soybean lecithin, phosphatidyl choline, cholesterol, cyclodextrins, isopropyl myristate, oleic acid, polysorbate 80, diethylene glycol monoethyl ether (Transcutol, Gattefossée, France).

Preferably, the film of the present invention is 20 to 500 μm thick.

It is a further object of the present invention to provide a process for the preparation of the aforesaid single-layer film supported by an antistick protective sheet, which comprises the following steps:

a) preparing a water solution of the film-forming agent;

b) adding the solution of step a) to a solution of the hydrophilic adhesive polymer;

c) adding one active ingredient at least, in the form of water solution or micronised particles or emulsion;

d) spreading the mixture obtained in step c) as a thin layer, preferably 50 to 1,000 μm thick, on an antistick sheet of plastic material or aluminium or paper coated with silicone or fluoropolymers (e.g. available from 3M, USA, or from Rexam Release, USA);

e) drying the layer obtained in step d) to residual humidity of 4% to 20%.

Drying is carried out by conventional methods, e.g. by oven or infra-red rays drying.

The single-layer film obtained, supported by an antistick protective sheet, may be opportunely divided into portions having the shape and surface suitable for the various therapeutic applications and may be suitably packaged, ready for use, in sterile air-tight packages.

Preferably, the mixture obtained in step c) consists of 0.1% to 20% active ingredient, 5% to 40% (w/w) film-forrning agent, 1% to 15% (w/w) adhesive polymer, and 50% to 85% water. Preferably, the film-forming agent/adhesive polymer ratio ranges between 2 and 7.

In step c) the adhesive/film-forming mixture is optionally added not only with the active ingredient but also with 0.5% to 20% (w/w) of one or more substances acting as absorption promoters and/or humectants and/or plasticisers.

Preferably, the mixture of step c)—to be adequately smeared—should have a viscosity of 1,000 to 50,000 mPa.s, measured at a 10 rpm flow gradient by a rotary viscosimeter, Viscostar (Fungilab, France) with head TR11.

Once step e) has been completed, the drug/adhesive/film-forming agent is thinned down in consistency; the film surface exposed to the air loses most of its adhesiveness.

The present invention substantially differs from the transdermal arrangements already known not only in the number of layers but also because the protective sheet does not cover the adhesive surface, but covers the opposite surface.

On application, the surface exposed to the air is maintained on the water- or saliva-moistened skin by applying a slight pressure for few seconds. Thanks to the presence of water, the surface in contact with the skin regains its-adhesiveness, and by removing the protective sheet, the drug/adhesive/film-forming layer is transferred onto the skin as a transparent film with a non-sticky surface (FIG. 2), which adheres to the skin firmly and integrally for at least 24 hrs. Adhesiveness is secured by the micro-moisture that forms, as a result of perspiration, between the skin and the film. Conversely, the moisture of the upper layer, initially present after the protective film removal, dries out by exposure to the air.

Since the film of the present invention conducts electricity, it can be advantageously used for active ingredients transdermal administration by iontophoretic applications, whereby the quantity of active ingredient that crosses the skin and reaches the systemic circulation increases.

The film of the present invention offers many advantages over the semisolid formulations or sticking plasters currently used for active ingredients dermal and transdermal administration.

In particular, compared with traditional dermal and transdermal sticking plasters, the single-layer film offers the advantages listed below:

1. it can be prepared by a simple and no expensive procedure, which, furthermore, does not require organic solvents;

2. it is thin and very flexible and, therefore, perfectly adapts itself to the skin wrinkles and lines; hence, the film surface in contact with the skin and, consequently, the active ingredient release increase considerably;

3. it can be easily handled as it is non-sticky in the dry state;

4. it is permeable by water with the result that it does not cause the occlusive effect typical of plasters;

5. it conducts electricity and, therefore, can be used for iontophoretic applications.

As concerns iontophoresis, the film of the invention offers the following advantages:

1. the active ingredient keeps in contact with the skin even once the iontophoretic application has been completed;

2. it allows a greater adherence to the skin during and after iontophoretic application;

3. it simplifies iontophoresis procedures and makes them fit for outpatient use.

The following examples are given further to illustrate the present invention. The scope of this invention is not, however, meant to be limited to the specific details of the examples.

### EXAMPLE 1

Preparation of a Single-Layer Film Containing Lidocaine Chlorhydrate

Polyvinyl alcohol (13.02 g) having a molecular weight of 72,000 Da and a hydrolysis degree of 86% to 89% was dispersed in water (49 ml), previously heated to 80° C. The resulting mixture was stirred to complete dissolution. Separately, for adhesive preparation, water (18.15 ml), previously heated to 78° C. to 82° C., was added with Eudragit E100 (4.3 g), lauric acid (2.48 g) and adipic acid (0.48 g). The mixture was stirred at a constant temperature for approx. 30 min, cooled to 60° C., and added with glycerin (1.57 g). In a separate vessel, lidocaine chlorhydrate (2 g) was dissolved in water (5 ml). The polyvinyl alcohol solution was then added, in the order, with the adhesive solution, lidocaine solution and glycerin (4 g).

The mass obtained was spread, in the form of a thin film (250 μm thick), on a silicone-coated paper sheet (“liner”) with doctor blade (BYK-Gardner, Silver Spring, USA). The resulting product was fed to an air-circulated oven at 60° C. for a period of 30 min. Once the treatment was complete, round portions (approx. 7 cm² each) were cut from the coated strip.

The single-layer film obtained was 40 μm thick and had a lidocaine content of 2 mg/portion, i.e. 0.3 mg/cm² or 74 mg/cm³/portion.

### EXAMPLE 2

Preparation of a Single-Layer Film Containing Acyclovir

Polyvinyl alcohol (18.6 g) having a molecular weight of 49,000 Da and a hydrolysis degree of 86% to 89% was dispersed in water (44 ml), previously heated to 80° C. The resulting mixture was stirred to complete dissolution. Separately, for adhesive preparation, water (18.2 ml), previously heated to 78° C.-82° C., was added with Eudragit E100 (4.3 g), lauric acid (2.48 g) and adipic acid (0.48 g). The mixture was stirred for approx. 30 min at constant temperature, cooled to 60° C., and added with glycerin (0.27 g). In a separate vessel, acyclovir (1.5 g) was dispersed in glycerin (4 ml). The polyvinyl alcohol solution was then added, in the order, with the adhesive solution, an acyclovir dispersion and 6.17 g of a 70% sorbitol solution.

In this case, the active ingredient (acyclovir) was dispersed in the form of particles in the adhesive/film-forming mixture.

The mass obtained was spread, in the form of a thin film (250 μm thick), on a silicone-coated sheet of polymeric material (“liner”) with doctor blade (BYK-Gardner, Silver Spring, USA). The resulting product was fed to an air-circulated oven at 60° C. for a period of 30 min. Once the treatment was complete, round portions (approx. 7 cm² each) were cut from the coated strip.

The single-layer film obtained was 40 μm thick.

### EXAMPLE 3

Preparation of a Single-Layer Film Containing 5-Methoxypsoralen

Polyvinyl alcohol (18.6 g) having a molecular weight of 49,000 Da and a hydrolysis degree of 86% to 89% was dispersed in water (44 ml). The resulting mixture was stirred to complete dissolution. Separately, for adhesive preparation, water (19.33 ml), previously heated to 78° C.-82° C., was added with Eudragit E100 (4.3 g), lauric acid (2.48 g) and adipic acid (0.48 g). The mixture was stirred for approx. 30 min at a constant temperature, cooled to 60° C., and added with glycerin (0.27 g). In a separate vessel, 5-methoxypsoralen (0.01 g), cholesterol (0.08 g) and lecithin (0.07 g) were dissolved in ethanol (2.72 g) and isopropyl myristate (0.93 g). The solution was added with water (3 g) to form an emulsion. The polyvinyl alcohol solution was then added, in the order, with the adhesive solution, the drug-containing emulsion and glycerin (3.73 g).

The mass obtained was spread, in the form of a thin film (300 μm thick), on a silicone-coated sheet (“liner”) with doctor blade (BYK-Gardner, Silver Spring, USA). The resulting product was fed to an air-circulated oven at 60° C. for a period of 30 min. Once the treatment was complete, round portions (approx. 7 cm² each) were cut from the coated strip.

The single-layer film obtained, 40 μm thick, had an active ingredient content of 10 μg/portion.

### EXAMPLE 4

Preparation of a Single-Layer Film Containing Ibuprofen Lysine

Polyvinyl alcohol (13.02 g) having a molecular weight of 72,000 Da and a hydrolysis degree of 86% to 89% was dispersed in water (49 ml) previously heated to 80° C. The resulting mixture was stirred to complete dissolution. Separately, for adhesive preparation, water (25 ml), previously heated to 80° C., was added with tragacanth (2.08 g). The mixture was stirred to complete dissolution. In a separate vessel, ibuprofen lysine (3 g) was dissolved in water (2 ml). The polyvinyl alcohol solution was then added, in the order, with the adhesive solution, an ibuprofen lysine solution and 5.9 g of a 70% sorbitol solution.

The mass obtained was spread, in the form of a thin film (300 μm thick), on a silicone-coated sheet of polymeric material (“liner”) with doctor blade (BYK-Gardner, Silver Spring, USA). The resulting product was fed to an air-circulated oven at 60° C. for a period of 30 min. Once the treatment was complete, round portions (approx. 7 cm² each) were cut from the coated strip.

The single-layer film obtained was 40 μm thick.

### EXAMPLE 5

Assessment of Active Ingredient Release In vivo

The in vivo active ingredient release from the film prepared as per Example 1 was evaluated on volunteers, 24 to 26 years of age, using the tape stripping technique, proposed by the US FDA for the determination of the bioavailability/bioequivalence of topical formulations (US FDA, Topical dermatological drug products, NDAs and ANDAs—In vivo bioavailability, bioequivalence, in vitro release and associated studies, CDAR, 1998).

This technique is based on the removal of small portions of stratum corneum by repeated applications of the adhesive tape to the skin and successive extraction and analysis of the active ingredient contained therein.

To go into details, single-layer film portions obtained as per Example 1 were applied to the volunteers' forearm moistened skin and maintained there, with or without iontophoretic applications, for a period of 30 min. After said period, they were removed and tape stripping was carried out. In case of application in the presence of iontophoresis, an electrocardiography electrode connected to the positive pole of a constant-intensity d.c. generator, was attached to the film. A current density of 0.5 mA/cm² was applied for 30 min.

For purpose of comparison, a commercial formulation consisting of 2.5% lidocaine chlorhydrate gel, in an amount of 15 mg/cm² (corresponding to 0.3 mg/cm² lidocaine) was applied to a different part of the same forearm for 30 min. After said period, the formulation was removed with moistened cotton-wool. Still for purpose of comparison, the film as per Example 1 was applied to non-moistened skin for 30 min. In both cases, tape stripping was performed.

To go into details, the adhesive tape was consecutively applied 15 times to the same skin area that had been in contact with the film or with the lidocaine-containing gel. Each adhesive tape was weighed before and after application: the quantity of stratum corneum removed every time was determined. The adhesive tapes taken from a single volunteer were collected, in sequence, five at a time, in a test tube. Therefore, three samples per volunteer were obtained for each type of application, i.e. the first consisted of adhesive tapes 1-5, the second of adhesive tapes 6-10, and the third of adhesive tapes 11-15, including stratum corneum fragments localised at a different distance from the surface. The lidocaine present in each sample was then extracted with an eluent (3 ml) and analysed by high-performance liquid chromatograhy, using 300×3.9 mm μ-Bondapak C18 (Waters) column (Millipore, Milford, United States). The eluent used was a mixture of acetonitrile (14 parts) and 0.05 M potassium phosphate (86 parts), pumped at a flow rate of 1 milliliter per minute and monitored by spectrophotometer at 216 nm.

The detected amount of lidocaine was normalised in respect of the amount of stratum corneum contained in each sample of adhesive tape.

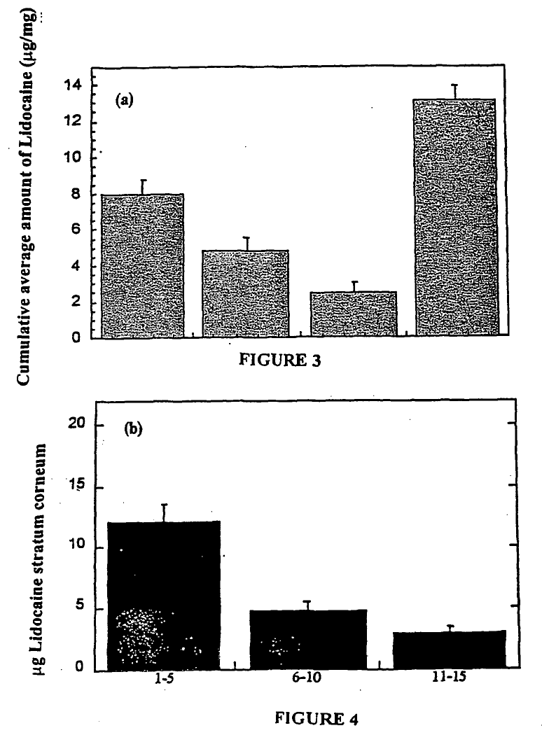

The results obtained are shown in FIGS. 3 and 4.

FIG. 3 shows the cumulative average amount of lidocaine per mg stratum corneum detected after application of the single-layer film to moistened skin (I); of a commercial formulation of lidocaine chlorhydrate (Luan®) (II); of the single-layer film to non-moistened skin (III); of single-layer film on moistened skin with iontophoretic application (IV). The data obtained prove that the single-layer film of the invention provides much higher active ingredient tissual concentrations than the traditional formulations and that said concentrations may be further increased by iontophoretic application. Furthermore, to adhere to the skin and release the drug appropriately, the film must be applied to moistened skin.

FIG. 4 shows the average distribution of lidocaine in the stratum corneum vs. the skin distance. As may be seen from the Figure, although lidocaine is especially present in the stratum corneum upper layers, non-negligible amounts also pass into the deeper layers.

1. Single-layer film for active ingredients dermal or transdermal administration comprising at least an active ingredient, a film-forming agent and a hydrophilic adhesive polymer.

2. Self-supporting water-permeable single-layer film for active ingredients dermal or transdermal administration comprising at least an active ingredient, a film-forming agent and a hydrophilic adhesive polymer.

3. The film as claimed in claims 1 and 2, wherein said active ingredient is in the dissolved or dispersed state.

4. The film as claimed in claims 1 and 2, wherein said film-forming agent is selected from the group consisting of ethylcellulose, acrylic and methacrylic polymers in an aqueous dispersion, and polyvinyl alcohol.

5. The film as claimed in claim 4, wherein said film-forming agent is polyvinyl alcohol having a molecular weight ranging from 500 to 100,000 Da.

6. The film as claimed in claim 5, wherein said polyvinyl alcohol has a molecular weight ranging from 49,000 to 72,000 Da.

7. The film as claimed in claim 5, wherein said polyvinyl alcohol has a hydrolysis degree ranging from 80% to 99%.

8. The film as claimed in claim 7, wherein said polyvinyl alcohol has a hydrolysis degree ranging from 85% to 89%.

9. The film as claimed in claims 1 and 2, wherein said hydrophilic adhesive polymer is selected from the group consisting of polyvinylpyrrolidone, tragacanth, gum arabic, karaya. xanthan gum, guar gum, acrylic and methacrylic adhesives, carrageenan and rosin.

10. The film as claimed in claim 9, wherein said hydrophilic adhesive polymer is a polyaminomethacrylate or tragacanth.

11. The film as claimed in claim 10, wherein said polyaminomethacrylate is Eudragit E100.

12. The film as claimed in claims 1 and 2, wherein said film-forming agent is polyvinyl alcohol having a molecular weight ranging from 500 to 100,000 Da and said hydrophilic adhesive polymer is a polyaminomethacrylate or tragacanth.

13. The film as claimed in claim 12, wherein said polyaminomethacrylate is Eudragit E100.

14. The film as claimed in claim 12, wherein said polyvinyl alcohol has a molecular weight ranging from 49,000 to 72,000 Da.

15. The film as claimed in claim 12, wherein said polyvinyl alcohol has a hydrolysis degree ranging from 80% to 99%.

16. The film as claimed in claim 15, wherein said polyvinyl alcohol has a hydrolysis degree ranging from 85% to 89%.

17. The film as claimed in claims 1 and 2 comprising at least a substance acting as an absorption promoter and/or humectant and/or plasticiser.

18. The film as claimed in claim 17, wherein said substance is selected from the group consisting of glycerin, ethyl alcohol, propylene glycol, polyethylene glycol having a molecular weight ranging from 400 to 6,000, sorbitol, phospholipids, soybean lecithin, phosphatidyl choline, cholesterol, cyclodextrins, isopropyl myristate, oleic acid, polysorbate 80, diethylene glycol monoethyl ether.

19. The film as claimed in claims 1 and 2 having a thickness of 20 to 500 μm.

20. Process for the preparation of a single-layer film as claimed in claims 1 and 2, comprising the following steps:
a) preparing a water solution of the film-forming agent;
b) adding the solution of step a) to a solution of the hydrophilic adhesive polymer,
c) adding one active ingredient at least;
d) spreading the mixture obtained in step c) as a thin layer, on an antistick sheet of plastic material or aluminium or paper coated with silicone or fluoropolymers;
e) drying the layer obtained in step d) to residual humidity of 4% to 20%.

21. The process as claimed in claim 20, wherein the layer obtained in step d) is 50 μm to 1,000 μm thick.

22. The process as claimed in claim 20, wherein the mixture obtained in step c) has a viscosity ranging from 1,000 to 50,000 mPa.s.

23. The process as claimed in claim 20, wherein the mixture obtained in step c) consists of 0.1% to 20% active ingredient, 5% to 40% (w/w) film-forming agent, 1% to 15% (w/w) adhesive polymer and 50% to 85% water.

24. The process as claimed in claim 23, wherein the film-forming agent/adhesive polymer ratio in said mixture ranges from 2 to 7.

25. The process as claimed in claim 20, wherein in said step c) said active ingredient is dissolved in the solution comprising the film-forming agent and the adhesive polymer.

26. The process as claimed in claim 20, wherein in said step c) said active ingredient is dispersed in the solution comprising the film-forming agent and the adhesive polymer in the form of micronised particles or of emulsion.

27. The process as claimed in claim 20, wherein in said step c) the solution comprising the film-forming agent and the adhesive polymer is added with 0.5% to 20% (w/w) at least of a substance acting as an absorption promoter and/or humectant and/or plasticiser.

28. Use of the single-layer film as claimed in claims 1 and 2 for active ingredients dermal or transdermal administration.

29. Use of the single-layer film as claimed in claims 1 and 2 for active ingredients iontophoretic application.
